(19) United States
(12) Patent Application Publication
(10) Pub. No.: US 2023/0293690 A1
(43) Pub. Date: Sep. 21, 2023

(54) 5' S/MAR APPLICATIONS

(71) Applicant: Deutsches Krebsforschungszentrum, Heidelberg (DE)

(72) Inventors: Richard Harbottle, Heidelberg (DE); Matthias Bozza, Heidelberg (DE)

(21) Appl. No.: 18/017,636

(22) PCT Filed: Jul. 21, 2021
(86) PCT No.: PCT/EP2021/070403

§ 371 Date: Jan. 23, 2023

(30) Foreign Application Priority Data
Jul. 22, 2020 (EP) 20187240.5

(51) Int. Cl. A61K 39/00; A61K 48/00; C12N 15/85

(52) U.S. Cl. CPC A61K 39/4632; A61K 39/4631; A61K 48/0066; C12N 15/85; C12N 2800/107

(57) ABSTRACT

The instant invention relates to a therapeutic cell comprising an episomal polynucleotide comprising a promoter and an expressible sequence, wherein said episomal polynucleotide further comprises an S/MAR element upstream of said promoter. The present invention further relates to expression constructs, polynucleotides, animal host cells, expression constructs, vectors, and/or polynucleotides comprising a cargo sequence related thereto.

The instant invention relates to a therapeutic cell comprising an episomal polynucleotide comprising a promoter and an expressible sequence, wherein said episomal polynucleotide further comprises an S/MAR element upstream of said promoter. The present invention further relates to expression constructs, polynucleotides, animal host cells, expression constructs, vectors, and/or polynucleotides comprising a cargo sequence related thereto.

Genetic modification of cells is used routinely in modern cell culture for scientific purposes. However, use of corresponding techniques in treatment of inherited diseases caused by mutations of genes, while being highly desirable, still is hampered by the problem that methods available usually only provide transient modification, such as transient transfection protocols, whereas methods providing stable modification of cells usually rely on integration of the transgene into the genome of the host cell. Integration of a transgene, however, even if targeted to a specific locus, bears the risk of inducing a deleterious mutation, which may lead e.g. to cancer as a side effect of treatment.

Scaffold/matrix attachment regions (S/MARs), which are also known as scaffold-attachment regions (SARs) or matrix-associated regions (MARs) are known as sequences in the genome of eukaryotic organisms mediating attachment of the nuclear matrix. Moreover, S/MAR sequences were found to have insulator properties, preventing extension of a condensed chromatin domain into a transcriptionally active region and the interaction of a distal enhancer with a promoter (Yusufzai & Felsenfeld (2004), PNAS 101(23), 8620). The S/MARS are AT-rich sequences, and some AT-rich motifs were found to be further enriched (Liebeich et al. (2002), NAR 30(15): 3433). In plants, S/MAR elements were found to expression of adjacent genes (Geest et al. (2004), Plant Biotech J 2:13).

A variety of vectors has been proposed for stable maintenance in cells based on S/MAR motifs, e.g. in U.S. Pat. No. 6,410,314 B1 and in Haase et al. (2010), BMC Biotechnology 10:20.

Transcription into the S/MAR element was found essential for stable maintenance of a corresponding vector (Rupprecht et al. (2010), Gene 466(1-2) 36) S/MAR sequences as part of a transcription unit, i.e. S/MAR sequences downstream of a promoter, were proposed e.g. in WO 2019/057773 A1, WO 2019/057774 A1, and WO 2019/060253 A1. Epigenetic effects having an influence on replication of S/MAR vectors were identified (Haase et al. (2013), PLOS One 8(11):e79262).

Nonetheless, improved S/MAR-based vectors being stable enough for use stable modification of cells, e.g. for gene therapy, are still desirable.

In accordance, the present invention relates to a therapeutic cell comprising an episomal polynucleotide comprising a promoter and an expressible sequence, wherein said episomal polynucleotide further comprises an S/MAR element upstream of said promoter.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A bas B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e., are equivalent to “comprising a least one”.

Further, as used in the following, the terms “preferably”, “more preferably”, “most preferably”, “particularly” “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a PH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result of use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr. Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis. As will be understood as well, the expression at least 98% includes the whole range of from 98% to 100%, including 100%.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin.

Unless specifically indicated otherwise herein, the compounds specified may be comprised in larger structures, e.g. may be covalently or non-covalently linked to carrier molecules, retardants, and other excipients. In particular, polynucleotides or expression constructs may be comprised in polynucleotides comprising further sequences, e.g. preferably, (further) selectable markers such as antibiotic resistance genes and/or sequences encoding marker polypeptides making the presence of a polynucleotide comprising said sequences detectable, and/or sequences ensuring propagation in bacterial cells, e.g. prokaryotic origins of replication. Thus, the polynucleotide or expression construct may in particular be comprised in a vector. Also, polypeptides as specified may be comprised in fusion polypeptides comprising further peptides, which may serve e.g. as a peptide tag for purification and/or detection, as a linker, or to extend the in vivo half-life of a compound. The term “peptide tag” refers to a stretch of amino acids which are added to or introduced into the polypeptide, preferably, the tag is added C- or N-terminally to the polypeptide of the present invention. Said stretch of amino acids preferably allows for detection of the fusion polypeptide by an antibody which specifically recognizes the tag; or it preferably allows for forming a functional conformation, such as a chelator, or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences.

The term “therapeutic cell”, as used herein, relates to a host cell as specified herein below comprising an expression construct as specified herein below and having the biological activity of being suitable for treatment and/or prevention of disease, i.e., preferably having the biological activity of ameliorating and/or preventing disease if administered in an effective dose to a subject suffering from said disease. The therapeutic cell may be any cell capable of receiving and stably episomally replicating the episomal polynucleotide and of expressing the expression construct comprised on the episomal polynucleotide. The therapeutic cell may in principle be xenogeneic; allogeneic, syngeneic, or autologous to its recipient, preferably is allogeneic, syngeneic, or autologous, more preferably is allogeneic or autologous to its recipient. More preferably, the therapeutic cell is allogeneic to its recipient, most preferably is autologous to its recipient. Preferably, the therapeutic cell is a cell of a subject as specified elsewhere herein, more preferably is a mammalian cell, most preferably a human cell. The therapeutic cell may, in principle, be any cell, preferably a somatic cell, deemed appropriate by the skilled person for the intended purpose. Thus, the therapeutic cell may be a blood cell, a neuron or glia cell, a skin cell, a muscle cell, a mucosa cell, a cell of an organ, such as a hepatic cell, a pancreatic cell, a lung cell, a kidney cell, a retina cell, an enteric cell, and the like, a connective tissue cell, or a bone cell. Preferably, the therapeutic cell is a cell from the tissue and/or organ to be treated, in particular in case the disease to be treated is a genetic disease; thus, e.g. in Choroideremia, the therapeutic cell may be a retina cell. Preferably, the therapeutic cell is not a germline cell, in particular is not an oocyte or spermatozoon of precursor cell thereof. Preferably, the therapeutic cell is a stem cell, preferably not produced by destruction of an embryo; thus, the stem cell preferably is a cell from an established stem cell line or a somatic stem cell (adult stem cell). Preferably, the therapeutic cell is not a totipotent stem cell. Preferably, the therapeutic cell is a pluripotent; multipotent, or an oligopotent stem cell. Also preferably, the therapeutic cell is a blood cell, in particular an immune cell, more preferably a T cell, a B ell, a dendritic cell, monocyte, a granulocyte, or & plasma cell, or a progenitor of any of the aforesaid. Preferably, the therapeutic cell is it CD34+ Progenitor Cell; a CD61+ Thrombocyte; a CD19+ B-Lymphocyte; a CDI4+ Monocyte; a CD15+ Granulocyte; a CD3+ Cytotoxic T-Lymphocyte, preferably also positive for CD8 and CD45; a CD3+ Helper T-Lymphocyte, preferably also positive for CD4 and CD45; a CD3+ activated T-Lymphocyte, preferably also positive for CD25 and CD45, a Tumor infiltrating Lymphocyte, or a Natural Killer (NK) cell. Preferably, the therapeutic cell is a cell for administration to a subject.

The term “host cell”, as used herein, relates to any cell capable of stably extrachromosomally maintaining an episomal polynucleotide, expression construct, polynucleotide, or vector, as specified herein. Thus, the episomal polynucleotide, expression construct, polynucleotide, or vector may be maintained episomally in an eukaryotic cell and/or as a plasmid in a prokaryotic cell. Thus, the host cell preferably is a bacterial cell, more preferably a cell of a laboratory strain of bacteria, more preferably an Escherichia coli cell. More preferably, the host cell is a eukaryotic cell, preferably a plant or yeast cell, e.g. a cell of a strain of baker's yeast, or is an animal cell. Still more preferably, the host cell is an animal cell, preferably an insect cell or a mammalian cell, in particular a livestock or companion animal cell, such as a chicken cell, a goose cell, a duck cell, a goat cell, a sheep cell, a cattle cell, a pig cell, a horse cell, a dog cell, a cat cell, a hamster cell, a rat cell, a mouse cell, a hamster cell, or a guinea pig cell. Even more preferably, the host cell is a human cell.

The term “episomal polynucleotide”, as used herein, relates to a polynucleotide having the components as specified and replicating episomally in a host cell, preferably in a therapeutic cell, more preferably stably replicating episomally. The term “episomal” replication is, in principle, known to the skilled person to relate to replication of a polynucleotide without being integrated into the cellular genome, i.e. without becoming covalently attached to the cellular genome. Thus, preferably, episomal replication of a polynucleotide is replication of said polynucleotide as an autonomous replication unit. Preferably, episomal replication is maintenance of the polynucleotide in the host cell in the form of a circularly closed double-stranded DNA molecule. As will be understood by the skilled person, the actual replication of said polynucleotide may involve other forms, e.g. in rolling circle replication. Episomal maintenance of circular DNA preferably is verified by a plasmid rescue procedure known to the skilled person; i.e. preferably, by preparing a lysate of host cells and transforming the DNA comprised therein into appropriate bacterial cells, e.g. E. coli cells; if a suitable number of bacterial colonies obtainable by said method comprises the circular DNA as a plasmid having the same restriction pattern and/or sequence as the original circular DNA, it is, preferably, assumed that the circular DNA was maintained episomally in the host cells. A further method of verifying episomal maintenance, which is also known to the skilled person, is DNA/DNA blotting (“Southern Blot” method); thus, preferably, total DNA of host cells is prepared and digested with one or more restriction enzyme(s); if in a Southern Blot using the original plasmid as a probe only bands corresponding be original polynucleotide DNA are visible, it is preferably concluded that the plasmid is maintained episomally. In accordance the term “replicating episomally”, as used herein, relates to the activity of a polynucleotide to induce production of at least two replicas of said polynucleotide in a host cell during a cell replication cycle while said polynucleotide is present in said cell as an autonomously replicating entity; and stable episomal replication is episomal replication to such an extent that the polynucleotide is still detectable in the host cell after at least 50 cell divisions, preferably after at least 100 cell divisions, more preferably, after at least 250 cell divisions, most preferably, after at least 500 cell divisions. Preferably, the aforesaid number of cell divisions is the average number of cell divisions for a population of cells. Preferably, from the episomal polynucleotide, no transcripts are produced which comprise at the same time the expressible sequence and the sequence of the promoter and/or of the S/MAR element.

The episomal polynucleotide comprises at least a promoter and an expressible sequence, in the order 5′-promoter-expressible sequence-3′, and an S/MAR element upstream of said promoter. Thus, the order is 5′-S/MAR element-promoter-expressible sequence-3′. The promoter of the episomal polynucleotide directs expression of the expressible sequence; thus, the distance between the last nucleotide of the promoter and the first nucleotide of the expressible sequence preferably is at most 1 kbp, more preferably at most 500 bp, even more preferably at most 250 bp, most preferably at most 100 bp. Preferably, the distance between the last nucleotide of the S/MAR element and the first nucleotide of the promoter is at most 5 kbp, more preferably at most 2 kbp, even lore preferably at most 1 kbp, still more preferably at most 500 bp, most preferably at most 250 bp. Preferably, (i) the promoter and the expressible sequence and/or (ii) the S/MAR element and the promoter are not intervened by a (further) expressible sequence. Preferably, on the episomal polynucleotide the promoter and the expressible sequence together form a eukaryotic expression construct. The episomal polynucleotide may optionally comprise further nucleic acid sequences, in particular expression control sequences modulating the expression of the expressible sequence in eukaryotic host cells, further expressible sequences or genes, e.g. marker genes, additional S/MAR sequences, selectable marker genes, and the like. Expression of the expressible sequence comprises transcription of the expressible sequence, preferably into a translatable mRNA or into a non-coding RNA. Regulatory elements ensuring expression in eukaryotic cells, preferably host cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Examples for regulatory elements permitting expression in eukaryotic host cells, in addition to promoters as specified herein below, are CMV-enhancer, SV40-enhancer or a globin intron in mammalian and other animal cells. Regulatory sequences preferred for expression of miRNAs or siRNAs are also known in the art. Moreover, inducible or cell type-specific expression control sequences may be comprised in an episomal polynucleotide. Inducible expression control sequences may comprise tet or lac operator sequences or sequences inducible by heat shock or other environmental or host factors, preferably being hormones or cytokines. Suitable expression control sequences are well known in the art. Besides elements which are responsible for the initiation of transcription, regulatory elements may also comprise transcription termination signals, such as the SV40-poly-A site or the tk-poly-A site, downstream of the polynucleotide. The episomal polynucleotide of the present invention preferably is devoid of a of a simian virus 40 (SV40) origin of replication, a bovine papillomavirus (BPV) origin of replication, and an Epstein-Barr virus (EBV) origin of replication, preferably is devoid of a polyomavirus origin of replication, a papillomavirus origin of replication, and a herpesvirus origin of replication; more preferably is devoid of an origin of replication of an eukaryote-infecting virus. More preferably, the episomal polynucleotide is devoid of any known eukaryotic origin of replication. However, preferably, the episomal polynucleotide further comprises a prokaryotic, preferably a bacterial, origin of replication, in particular an E. coli origin of replication. Preferably, the prokaryotic origin of replication is the only origin of replication comprised in the episomal polynucleotide.

The term “promoter” is, in principle, known to the skilled person as a genetic element directing, optionally in concert with further regulatory elements, the level of transcription of a gene. A promoter may be constitutive, Le providing a constant level of transcription essentially independent of a host cell's state, or may be regulated, i.e. provide levels of transcription in dependence of a host cell's state. Moreover, a promoter may be cell type and/or tissue specific, i.e. provide a detectable level of transcription only in a few or only one cell type(s). Preferably, the promoter according to the present invention is active in the host cell as specified herein above, more preferably in the therapeutic cell as specified herein above. As will be understood by the skilled person, the selection of a promoter may depend on the type of host cell or therapeutic cell intended for targeting, suitable promoters for specific cell types as well as constitutive promoters are known in the art. Preferably, the promoter is a eukaryotic promoter, more preferably a constitutive eukaryotic promoter, even more preferably a strong eukaryotic promoter. More preferably, the promoter is a mammalian promoter, still more preferably a human promoter. Preferably, the promoter is an EF 1alpha (elongation factor 1 alpha) promoter, an UbiC (ubiquitin C) promoter, a ROSA 26 promoter, a PGK (phosphoglycerate kinase) promoter, and/or a CAG (chicken alpha-actin) promoter, more preferably is an EF1alpha promoter. Also preferably, the promoter is a cell- and/or tissue-specific eukaryotic promoter, As used herein, the term promoter is used for the promoter as specified above, whereas any other promoter potentially present on an expression construct, polynucleotide, or vector in addition is referred to as “secondary promoter”. Thus, preferably, the promoter is a promoter directing transcription away from the S/MAR sequence in a host cell; also preferably, a promoter not intervening an S/MAR sequence and an expressible sequence, e.g. being a prokaryotic promoter, a promoter having only a 3′ S/MAR sequence, and/or being a promoter directing transcription into the S/MAR sequence, is a secondary promoter.

The term “expressible sequence” is understood by the skilled person be a shorthand expression for the term “expressible nucleic acid sequence” and to relate to each and any nucleic acid sequence from which a gene product can be produced by a host cell, preferably by a therapeutic cell. Said gene product preferably is an RNA or a polypeptide, more preferably is a polypeptide. Thus, the expressible sequence, preferably, is a coding sequence, more preferably comprising an open reading frame. Preferably, the expressible sequence encodes a therapeutic gene product, i.e. a therapeutic RNA or a therapeutic polypeptide, more preferably a therapeutic polypeptide.

The term “therapeutic”, as used herein in the context of a therapeutic gene product, i.e. a therapeutic RNA or a therapeutic polypeptide, relates to a gene product contributing to treatment and/or prevention of disease as specified herein below. Thus, “contributing to” treatment and/or prevention of disease, preferably, relates to causing a statistically significant ameliorative and/or preventive effect, preferably when administered at an effective dose.

Preferably, the therapeutic gene product is a therapeutic RNA, the term “therapeutic RNA” relating to any RNA mediating a change in a physiological and/or metabolic state of a host cell comprising said therapeutic RNA. Preferably, the therapeutic RNA mediates a change in a physiological and/or metabolic state of a host cell comprising said therapeutic RNA, thereby contributing to amelioration or treatment of a disease or disorder, preferably as specified elsewhere herein. The therapeutic RNA may be an mRNA encoding a therapeutic polypeptide as specified herein below or a subunit or active fragment thereof. More preferably, the therapeutic RNA is a non-coding RNA; non-coding RNAs are widely known in the art and are in particular used for reducing or abolishing expression of a gene of interest. Thus, preferably, a therapeutic RNA is an interfering, non-coding RNA, in particular an siRNA, a miRNA, an antisense RNA, or a ribozyme. Means and methods for designing interfering nucleic acids for use in e.g. gene silencing, are known to the skilled person. Requirements for an RNA as described to be effective in inhibiting expression of a target gene are also known in the art. The therapeutic gene product may also be a guide RNA, i.e. an RNA used to guide a CRISPR/Cas nuclease to a target gene of interest. The therapeutic gene product may also be a decoy RNA, modulating expression of a target gene by providing additional binding sites for a regulatory polypeptide regulating expression of said target gene. Moreover, the therapeutic gene product may also be an miRNA, which are known in the art to provide for global gene regulation. Thus, the therapeutic RNA preferably is an SiRNA, an shRNA, an antisense RNA, a gRNA, a ribozyme, a decoy RNA, or an miRNA, more preferably is an siRNA, an shRNA, or an antisense RNA.

More preferably, the therapeutic gene product is a therapeutic polypeptide. As used herein, the term “therapeutic polypeptide” relates to any polypeptide mediating a change in a physiological and/or metabolic state of a host cell comprising said therapeutic polypeptide and/or of cells being in direct or in fluid contact with such host cell, preferably via a bodily fluid, more preferably via blood, lymph, saliva, cerebrospinal fluid, and/or interstitial fluid. More preferably, the therapeutic polypeptide is a polypeptide mediating a change in a physiological and/or metabolic state of a host cell and/or of cells being in direct or in fluid contact with such host cell, thereby contributing to amelioration or treatment of a disease or disorder, preferably as specified elsewhere herein. Preferably, the therapeutic polypeptide is an antibody, preferably as specified herein below. Also preferably, the therapeutic polypeptide is a T Cell Receptor (TCR), more preferably a human or chimeric T Cell receptor, a Chimeric Antigen Receptor (CAR), preferably MART1 TCR, a cytokine, and/or a polypeptide lacking in cells affected with a genetic disease as specified elsewhere herein. Preferably, the CAR and/or the T-cell receptor is tumor antigen specific. Thus, e.g. preferably, the polynucleotide comprises at least one expressible construct encoding a polypeptide providing phenylalanine-hydroxylase activity (EC 1.14.16.1) for treatment of phenylketonuria, or encoding the REP1 gene for treating Choroideremia, or encoding the RPE65 gene for treating Leber's congenital amorosis, or encoding Factors VIII, IX and/or X for treatment of Haemophilia, or encoding the USH2a gene for treating Ushers disease.

The term “antibody” is used herein in the broadest sense and specifically covers monoclonal antibodies, multispecific antibodies (e.g. bispecific antibodies) formed from at least two intact antibodies, single-chain antibodies, single-domain antibodies (VHH), also known as nanobodies, and antibody fragments as long as they exhibit the desired binding activity. Preferably, the antibody is a single-chain antibody or a VHH (nanobody). Preferably, the antibody is a therapeutic antibody, i.e. has binding activity for a disease-related molecule, preferably a polypeptide of therapeutic relevance and contributes to treatment of a disease or disorder caused or aggravated by said disease-related molecule. “Antibody fragments”, as related to herein, comprise a portion of an intact antibody comprising the antigen-binding region thereof. Examples of antibody fragments include Fab, Fab′, F(ab′)2, and Fv fragments; diabodies, linear antibodies; single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. “Fv” is the minimum antibody fragment which contains a complete antigen-binding site. Preferably, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain FV species. It is in this configuration that the three hypervariable regions (HVRs, also referred to as complementarity determining regions (CDRs)) of each variable domain interact to define an antigen-binding site. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site. The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Preferably, the antibody is an anti-tumor antigen antibody. more preferably an anti-tumor specific antigen antibody, even more preferably an anti-carcinoembryonic antigen (CEA) antibody, still more preferably a single-chain anti-CEA antibody.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99% The p-values are, preferably, 0.1, 0.05, 0.01, 0.005, or 0.0001. Preferably, the treatment shall be effective for at least 10%, at least 20% at least 50%, at least 60%, at least 70%, at least 80%, or at least 90% of the subjects of a given cohort or population. As will be understood by the skilled person, effectiveness of treatment of disease, e.g. cancer, is dependent on a variety of factors including, e.g. disease stage and disease type. Preferably, treating cancer is reducing tumor burden or keeping tumor burden constant in a subject. Thus, preferably, treating has the effect of causing a tumor to stop growing, more preferably to cause regression of a tumor, more preferably of causing a tumor to resolve.

The term “preventing” refers to retaining health with respect to the diseases or disorders referred to herein for a certain period of time in a subject. It will be understood that said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification.

The term “S/MAR element”, also known under the designation “scaffold/matrix attachment region”, is, in principle, known to the skilled person to relate to a DNA sequence mediating attachment of the nuclear matrix of a eukaryotic cell to said DNA. S/MAR sequences typically are derived from sequences in the DNA of eukaryotic chromosomes. A variety of S/MAR sequences is available, and sequences are available from public databases, e.g. as described in Liebich et al. (2002), Nucleic Acids Res. 30, 312-374. According to the present invention, the nucleic acid sequence of said S/MAR element (referred to as “S/MAR sequence”) preferably comprises a nucleic acid sequence being at least 70% more preferably at least 80%, even more preferably at least 90%, still more preferably at least 93%, still more preferably at least 9596, most preferably at least 98%, identical to SEQ ID NO:1 and/or comprises a nucleic acid sequence being at least 70%, more preferably at least 80%, even more preferably at least 90%, still more preferably at least 93%, still more preferably at least 95%, most preferably at least 98%, identical to SEQ ID NO:2, more preferably comprises a nucleic acid sequence being at least 70%, more preferably at least 80%, even more preferably at least 90%, still more preferably at least 93%, still more preferably at least 95%, most preferably at least 98%, identical to SEQ ID NO:1. Preferably, the S/MAR element comprises a sequence at least 70%, more preferably at least 80%, even more preferably at least 90%, still more preferably at least 93%, still more preferably at least 95%, even more preferably at least 97%, even more preferably at least 98%, most preferably at least 99%, identical to SEQ ID NO:3. Also preferably, the S/MAR element comprises sequence at least 70%, more preferably at least 80%, even more preferably at least 90%, still more preferably at least 93% still more preferably at least 95%, most preferably at least 98%, identical to Genbank Acc. No. AY220727.1 (SEQ ID NO:4).

Preferably, the episomal polynucleotide comprises or further comprises a selectable marker gene, in particular coding for a selectable marker polypeptide. As used herein, the term “selectable marker gene” is used as a shorthand for the expression “expressible coding sequence encoding a selectable marker polypeptide”. The term “selectable marker” is in principle understood by the skilled person and relates to a nucleic acid sequence conferring, when expressed in a host cell, resistance to at least one condition mediating selective pressure to a host cell when applied thereto. Selectable markers are known in the art for prokaryotic and for eukaryotic cells. Preferably, the selectable marker is a selectable marker of a eukaryotic cell. Preferably, the selectable marker is a selectable marker polypeptide, more preferably a selectable marker polypeptide having transporter and/or enzymatic activity removing a selective compound from a host cell or modifying said selective compound to make it inactive. Preferably, the selectable marker is a marker mediating resistance to puromycin, to blasticidin, neomycin, and/or to zeocin, more preferably to puromycin. Thus, preferably, the episomal polynucleotide comprises a secondary promoter and the selectable marker, which together constitute e.g. a puromycin resistance gene, a blasticidin resistance gene, a neomycin resistance gene, or a zeocin resistance gene, more preferably a puromycin resistance gene. The selectable marker sequence may, however, also be part of the expressible sequence as specified above, the selectable marker sequence being the expressible sequence, being part of a fusion polypeptide encoded by the expressible sequence, or being expressed as a discrete polypeptide, e.g. via a ribosomal re-entry site. Preferably, the selectable marker is a polypeptide providing resistance to a specific set of growth conditions, preferably presence and/or absence of proliferation signals. Thus, in a preferred embodiment, the selectable marker is a T-cell receptor (TCR) or a chimene antigen receptor (CAR), both of which are in principle known in the art, preferably being those as specified elsewhere herein. Preferably, the TCR and/or the CAR have a known specificity, such that, preferably, T cell signaling can be induced in host cells comprising said TCR and/or CAR. Preferably, in such case, the host cell is a T cell or an NK cell. Thus, the promoter and the expressible sequence as specified elsewhere herein may together constitute a selectable maker gene.

Advantageously, it was found in the work underlying the present invention that a polynucleotide comprising a promoter and an expressible sequence, wherein said polynucleotide further comprises an S/MAR element upstream of said promoter is maintained episomally in a eukaryotic cell, i.e. is not integrated into the genome of the host cell. Thus, a host cell comprising an episomal polynucleotide of the present invention is genetically more clearly defined and/or genetically more stable than comparable host cells comprising integrated copies of an expression construct, making these host cell described herein particularly suitable as therapeutic cells, since the risk of inadvertent genetic modification is reduced.

The definitions made above apply mutatis mutandis to the following. Additional definitions and explanations made further below also apply for all embodiments described in this specification mutatis mutandis.

The present invention also relates to the therapeutic cell as specified herein for use as a medicament, in particular for use in immunotherapy for treatment of genetic disease in a subject.

Preferably, the aforesaid uses comprise administration of an effective dose of therapeutic cells and/or animal host cells as specified herein to a subject; more preferably, immunotherapy and/or treatment of genetic disease comprises administration of at least 10%, preferably at least 10⁶, more preferably at least 10⁷ of said therapeutic cells or animal host cells to a subject. As will be understood, the therapeutic cells, preferably at the aforesaid dose, may be administered more than once, e.g. preferably at least twice, more preferably at least three times, even more preferably at least five times, most, preferably at least ten times. Also preferably, the aforesaid uses comprise maintenance of said therapeutic cells for at least 1 month, preferably at least 6 months, more preferably at least one year, preferably in the body of said subject, the term “maintenance” relating to continued existence in a detectable state and number.

The term “subject”, as used herein, relates to an animal, preferably a vertebrate, more preferably a mammal, in particular to livestock like cattle, horse, pig, sheep, and goat, or to a laboratory animal like a rat, mouse, and guinea pig. Most preferably, the subject is a human. Preferably, the subject is at risk to suffer from, is expected to suffer from, or is suffering from a disease as related to herein, preferably cancer autoimmune disease, and/or genetic disease.

The term “cancer”, as referred to herein, refers to a disease of an animal, including man, characterized by uncontrolled growth by a group of body cells (“cancer cells”). This uncontrolled growth may form a mass of cancer cells (“tumor”), which may be accompanied by intrusion into and destruction of surrounding tissue (“invasion”) and possibly spread of cancer cells to other locations in the body (“metastasis”). Moreover, cancer may entail recurrence of cancer cells after an initial treatment apparently removing cancer cells from a subject (“relapse”). As used herein, the term cancer cells preferably includes cancer stem cells. Preferably, the cancer is selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, cancer, medulloepithelioma; melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small lung cancer, oral cancer, oropharyngeal cancer, osteosarcoma, ovarian cancer, ovarian epithelial cancer, ovarian germ cell tumor, ovarian low malignant potential tumor, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, skin cancer, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenström macroglobulinemia, and wilms tumor.

The term “autoimmune disease”, as used herein, refers to a disease that arises from an abnormal immune response of a subject's immune system against substances and tissues normally present in the body of said subject. Autoimmunity may affect the whole organism, may be restricted to certain organs, or may involve a particular tissue type, possibly in different locations of the body. The diagnosis of an autoimmune disease is based on an individual's symptoms, findings from a physical examination, and results from laboratory tests. Typical tests for autoimmune diseases are known in the art and include blood tests, urine tests, swabs, diagnostic tests, lab tests, and pathology testing. However, some autoimmune diseases may be difficult to diagnose, especially in the early stages of the disease. Preferably, the autoimmune diseases is systemic lupus erythematosus (SLE), sarcoidosis, scleroderma, rheumatoid arthritis, Diabetes mellitus type 1, autoimmune thyroiditis (e.g. Hashimoto's thyroiditis), Addison's disease, and multiple sclerosis.

The term “genetic disease”, as used herein, relates to a disease causally linked to one or more modifications, preferably mutations, in the genome of a subject. Thus, preferably, the genetic disease is causally linked to one or more epigenetic changes, more preferably is causally linked to one or more genetic mutations. As will be understood, symptoms of a genetic disease often are caused by expression of a mutated gene and/or lack of expression of a gene providing normal function of the gene product in one or more specific tissue(s) and/or cell type(s). Thus, it may be preferable to treat genetic disease only in those cells in which the mutation contributes to disease. Preferably, the genetic disease is a monogenic disease, i.e. is caused by a genetic alteration in one gene. More preferably, the genetic disease is a monogenic recessive disease, i.e. is caused by generic alterations in both alleles of a gene; thus, preferably, the amelioration of symptoms is expected by provision of at least one unaltered copy of the affected gene. Most preferably, the genetic disease is phenylketonuria, alkaptonuria, Leber's Congenital Amaurosis, Choroideremia, or Stargardt disease. In a preferred embodiment, the genetic disease is cancer.

The term “immunotherapy”, as used herein, relates to the treatment and/or prevention of disease, preferably of cancer and/or autoimmune disease, by modulation of the immune response of a subject. Said modulation may be inducing, enhancing, or suppressing said immune response, preferably by administration of a therapeutic cell, episomal polynucleotide, or expression construct as specified herein. Said immunotherapy may further comprise, e.g. administration of at least one cytokine, and/or of at least one antibody specifically recognizing e.g. cancer cells. The term “cell based immunotherapy” relates to a therapy comprising application of immune cells, e.g. T-cells, preferably tumor-specific NK cells, to a subject. Preferably said immune cells are therapeutic cells as specified herein above, in particular therapeutic cells comprising an expressible sequence encoding a T cell receptor, preferably a CAR.

The instant invention further relates to a polynucleotide comprising an S/MAR element, wherein said S/MAR element comprises a nucleic acid sequence being at least 93%, preferably at least 95%, identical to SEQ ID NO:3.

In the polynucleotide, the S/MAR element comprises a nucleic acid sequence being at least 93%, preferably at least 956, more preferably at least 96%, even more preferably at least 97%, still more preferably at least 98%, still more preferably at least 99%, most preferably 100%, identical to SEQ ID NO:3. More preferably, the S/MAR element consists of a nucleic acid sequence being at least 93%, preferably at least 95%, more preferably at least 96%, even more preferably at least 97%, still more preferably at least 98%, still more preferably at least 99%, most preferably 100%, identical to SEQ ID NO:3.

Preferably, the polynucleotide comprising an S/MAR element further comprises a cargo sequence. As used herein, the term “cargo sequence” relates to any sequence deemed desirable to the skilled person for being introduced into a host cell. Thus, the cargo sequence may be a a nucleic acid sequence comprising a gene or part thereof, preferably in its natural configuration. Thus, the cargo sequences may comprise regulatory sequences such as promoters, transcription factor binding sites, termination sites, enhancers, and the like. The cargo sequence may also comprise the natural intron/exon structure of a gene. Preferably, the cargo sequence comprises at least an expressible sequence as specified herein above, more preferably with an upstream promoter.

The instant invention also relates to an expression construct comprising an expressible sequence and a promoter, wherein said expression construct further comprises an S/MAR element upstream of said promoter, and wherein said expression construct replicates episomally in a mammalian cell.

The terms “promoter”, “expressible sequence”, “S/MAR element”, and “episomal replication” have been defined herein above.

The polynucleotide and the expression construct of the present invention preferably are devoid of a simian virus 40 (SV40) origin of replication, a bovine papillomavirus (BPV) origin of replication, and an Epstein-Barr virus (EBV) origin of replication, preferably is devoid of a polyomavirus origin of replication, a papillomavirus origin of replication, and a herpesvirus origin of replication; more preferably are devoid of an origin of replication of an eukaryote-infecting virus. More preferably, polynucleotide and the expression construct preferably are devoid of any known eukaryotic origin of replication. However, preferably, the polynucleotide and the expression construct may further comprise a prokaryotic, preferably a bacterial, origin of replication, in particular an E. coli origin of replication. Preferably, the prokaryotic origin of replication is the only origin of replication comprised in the polynucleotide and/or the expression construct.

The present invention also relates to a polynucleotide as specified herein above and/or an expression construct as specified herein above for use as a medicament, and/or for use in immunotherapy and/or treatment of genetic disease in a subject.

The present invention further relates to a vector comprising the polynucleotide according to the present invention and/or the expression construct according to the present invention.

The term “vector”, as used herein, relates to a polynucleotide comprising at least one nucleic acid sequence permitting propagation, amplification, and/or packaging of the polynucleotide in a host cell, preferably a non-therapeutic host cell. Preferably, the term encompasses phage, plasmid, viral or retroviral vectors as well artificial chromosomes, such as bacterial or yeast artificial chromosomes. In accordance, the vector may be a plasmid vector which can be propagated and amplified in a bacterial cell; in accordance, the plasmid vector preferably comprises a bacterial origin of replication. The vector may also be a viral vector, which may be propagated, amplified and/or packaged in a permissive cell or a packaging cell line. Thus, the viral vector may comprise sequences required for viral replication and/or packaging.

Preferably, cells permissive for viral vector replication and/or packaging cell lines are not therapeutic cells. The vector may also be an artificial chromosome of a non-therapeutic cell, e.g. of a bacterial cell, of a yeast cell, or of an insect cell. Appropriate sequences are known in the art for the respective vectors. The vector may be introduced into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. In a preferred embodiment, the vector is a bacterial vector, preferably having a p15A origin of replication and/or carrying a kanamycin resistance gene. Methods which are well known to those skilled in the art can be used to construct recombinant vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

The present invention also relates to a polynucleotide comprising a cargo sequence and an S/MAR element, wherein said polynucleotide is maintained episomally in a mammalian host cell and wherein said S/MAR element is transcribed at a frequency of at most 10 detectable copies per cell, preferably at most 1 detectable copies per cell, still more preferably at most 0.1 copies per cell, most preferably at an undetectably level.

The term “cargo sequence” has been specified herein above. The polynucleotide comprising a cargo sequence is maintained episomally in an animal host cell, preferably a mammalian host cell, as specified herein above.

In the polynucleotide comprising a cargo sequence, the S/MAR sequence is transcribed at a frequency of at most 10 detectable copies per cell, preferably at most 1 detectable copies per cell, still more preferably at most 0.1 copies per cell, most preferably at an undetectably level, means and methods for determining the number of detectable copies of an RNA per cell are known in the art; preferably, said method is qPCR, more preferably RT-qPCR, preferably with a detection limit of 0.01 copies per cell. Preferably, the S/MAR element in the polynucleotide comprising a cargo sequence is positioned such that any promoter comprised in the cargo sequence directs transcription away from the S/MAR element; thus, the S/MAR element is preferably positioned upstream of any promoter comprised in the cargo sequence.

The present invention also relates to an animal host cell comprising an S/MAR element, wherein said S/MAR element comprises a nucleic acid sequence being at least 70% identical to SEQ ID NO:1 and/or a nucleic acid sequence being at least 70% identical to SEQ ID NO:2.

The present invention also relates to the animal host cell of the present invention for use as a medicament, for use in immunotherapy and/or treatment of genetic disease in a subject.

The instant invention also relates to the therapeutic cell, the expression construct, the polynucleotide, the animal host cell, the expression construct, the vector, and/or the polynucleotide comprising a cargo sequence of the present invention for use in the manufacture of a medicament, in particular for use in immunotherapy and/or treatment of genetic disease.

The present invention also relates to a method for treating a subject suffering from a disease as specified herein, preferably cancer, autoimmune disease, and/or genetic disease, comprising contacting said subject with a therapeutic cell, expression construct, polynucleotide, animal host cell, expression construct, vector, and/or polynucleotide comprising a cargo sequence of the present invention and, thereby, treating said disease.

In view of the above, the following embodiments are particularly envisaged:
1. A therapeutic cell comprising an episomal polynucleotide comprising a promoter and an expressible sequence, wherein said episomal polynucleotide further comprises an S/MAR element upstream of said promoter.
2. The therapeutic cell of claim 1, wherein said expressible sequence encodes a therapeutic gene product, preferably a therapeutic polypeptide.
3. The therapeutic cell of claim 1 or 2, wherein said therapeutic polypeptide is an antibody, a T Cell Receptor (TCR), a Chimeric Antigen Receptor (CAR), a cytokine, and/or a polypeptide lacking in cells affected with a genetic disease.
4. The therapeutic cell of any one of claims 1 to 3, wherein said CAR and/or said T-cell receptor is tumor antigen specific.
5. The therapeutic cell of any one of claims 1 to 4, wherein said episomal polynucleotide comprises a selectable marker gene.
6. The therapeutic cell of any one of claims 1 to 5, wherein said S/MAR element comprises a nucleic acid sequence being at least 70% identical to SEQ ID NO:1 and/or a nucleic acid sequence being at least 70% identical to SEQ ID NO:2, preferably a nucleic acid sequence at least 70% identical to SEQ ID NO:3.
7. An animal host cell comprising an S/MAR element, wherein said S/MAR element comprises a nucleic acid sequence being at least 70% identical to SEQ ID NO:1 and/or a nucleic acid sequence being at least 70% identical to SEQ ID NO:2.
8. The animal host cell of claim 7, wherein said host cell is a therapeutic cell.
9. The animal host cell of claim 7 or 8, wherein said S/MAR element is comprised in an episomal polynucleotide.
10. The animal host cell claim 9, wherein said episomal polynucleotide comprises at least one feature as specified in any one of claims 1 to 6.
11. A therapeutic cell according to any one of claims 1 to 6 or an animal host cell according to any one of claims 7 to 10, for use as a medicament, in particular for use in immunotherapy and/or treatment of genetic disease in a subject.
12. The therapeutic cell for use or the animal host cell for use of claim 11, wherein said immunotherapy is treatment and/or prevention of cancer and/or autoimmune disease.
13. The therapeutic cell for use or the animal host cell for use of claim 11 or 12, immunotherapy and/or treatment of genetic disease comprises administration of at least 105, preferably at least 106, more preferably at least 107 of s therapeutic cells or animal host cells to a subject.
14. The therapeutic cell for use or the animal host cell for use of any one of claims 11 to 13, wherein said immunotherapy and/or treatment of genetic disease comprises maintenance of said therapeutic cells or animal host cells for at least 1 month, preferably at least 6 months, more preferably at least one year.
15. The therapeutic cell for use or the host cell for use of any one of claims 11 to 14, wherein said treatment comprises repeated ad ministration of said therapeutic cells or host cells.
16. The therapeutic cell for use of the animal host cell for use of any one of claims 11 to 15, wherein said therapeutic cell or host cell is a syngenic cell, preferably an autologous cell of said subject.
17. The therapeutic cell for use or the animal host cell for use of any one of claims 11 to 16, wherein said therapeutic cell or host cell is not rejected by a recipient's immune system.
18. The subject matter of any of the preceding claims, wherein said S/MAR element comprises a nucleic acid sequence being at least 70% identical to SEQ ID NO:2.
19. The subject matter of any of the preceding claims, wherein said S/MAR element comprises a nucleic acid sequence being at least 70% identical to SEQ ID NO:3.
20. The subject matter of any of the preceding claims, wherein said S/MAR element is maintained episomally in said therapeutic or host cell.
21. The subject matter of any of the preceding claims, wherein said therapeutic cell or host cell is a vertebrate cell, preferably a mammalian cell, more preferably a human cell.
22. The subject matter of any of the preceding claims, wherein said therapeutic cell or host cell is an immune cell, preferably, a CD34+ Progenitor Cell; a CD61+ Thrombocyte; a CD19+ B-Lymphocyte; a CD14+ Monocyte; a CD15+ Granulocyte; a CD3+ Cytotoxic T-Lymphocyte, preferably also positive for CD8 and CD45; a CD3+ Helper T-Lymphocyte, preferably also positive for CD4 and CD45; & CD3+ activated T-Lymphocyte, preferably also positive for CD25 and CD45, a Tumor infiltrating Lymphocyte, or a Natural Killer (NK) cell.
23. A polynucleotide comprising an S/MAR element, wherein said S/MAR element comprises a nucleic acid sequence being at least 93%, preferably at least 95%, identical to SEQ ID NO:3.
24. The polynucleotide of claim 23, further comprising an expressible sequence and a promoter, preferably a mammalian promoter, wherein said S/MAR sequence is located upstream of said promoter.
25. The polynucleotide of claim 23 or 24, wherein said expressible sequence encodes a therapeutic gene product, preferably a therapeutic polypeptide.
26. The polynucleotide of any one of claims 23 to 25, further comprising a selectable marker gene.
27. The polynucleotide according to any one of claims 23 to 26 and/or the vector according to claim 27, for use as a medicament, in particular for use in immunotherapy and/or treatment of genetic disease in a subject.
28. An expression construct comprising an expressible sequence and a promoter, wherein said expression construct further comprises an S/MAR element upstream of said promoter, and wherein said expression construct replicates episomally in a mammalian cell.
29. The expression construct of claim 28, wherein said expression construct is devoid of a simian virus 40 (SV40) origin of replication, a bovine papillomavirus (BPV) origin of replication, and an Epstein-Barr virus (EBV) origin of replication, preferably is devoid of a polyomavirus origin of replication, a papillomavirus origin of replication, and a herpesvirus origin of replication; more preferably is devoid of an origin of replication of an eukaryote-infecting virus, preferably a mammal-infecting virus.
30. The expression construct of claim 28 or 29, wherein said expressible sequence encodes a therapeutic polypeptide.
31. The expression construct of any one of claims 28 to 30, wherein said therapeutic polypeptide is an antibody, a T Cell Receptor (TCR), a Chimeric Antigen Receptor (CAR), a cytokine, and/or a polypeptide lacking in cells affected with a genetic disease.
32. The expression construct of claim 31, wherein said CAR and/or said T-cell receptor is tumor antigen specific.
33. The expression construct of any one of claims 28 to 32, comprising, preferably consisting of, the polynucleotide according to any one of claims 24 to 25.
34. A vector comprising the polynucleotide according to any one of claims 23 to 26 and/or the expression construct according to anyone of claims 28 to 33.
35. A polynucleotide comprising a cargo sequence and an S/MAR element, wherein said polynucleotide is maintained episomally in an animal host cell and wherein said S/MAR element is transcribed at a frequency of at most 10 detectable copies per cell, preferably at most 1 detectable copies per cell, still more preferably at most 0.1 copies per cell, most preferably at an undetectably level. (qPCR)
36. The subject matter of any of the preceding claims, wherein said S/MAR element is a heterologous S/MAR element.
37. The subject matter of embodiment 27 for use in immunotherapy, wherein said immunotherapy is treatment and/or prevention of cancer and/or autoimmune disease by modulation of the immune response of a subject.
38. The subject matter of embodiment 27 for use in treatment of genetic disease, wherein the genetic disease is a monogenic recessive disease, preferably is phenylketonuria, alkaptonuria, Leber's Congenital Amaurosis, Choroideremia, or Stargardt disease.

All references cited in this specification are herewith incorporated by reference with respect to their entire disclosure content and the disclosure content specifically mentioned in this specification.

### FIGURE LEGENDS

FIG. 1: Colony forming assay with S/MAR constructs; 1=GFP-β-interferon MAR, 2=Mar1 (SEQ ID NO:5)-GFP, 3=Mar2 (SEQ ID NO:3)-GFP, 4=Mar3 (SEQ ID NO:6)-GFP, 5-Mar4 (SEQ ID NO.7)-GFP; y-Axis number of resistant colonies.

FIG. 2: Number of transgene expressing (GFP+) cells and medium fluorescent intensity (MFI) of cell populations carrying various constructs, 1=unmodified cells, 2=GFP-β-interferon MAR, 3=Mar2-GFP.

FIG. 3: Detection of episomal replication by Southern blot; a=Mar2-GFP control vector, b=total DNA extracted from Hek293T established with Mar2-GFP; the GFP gene was detected.

FIG. 4: Nucleic acid sequences of nt 220 to 382 of Mar2 (1, SEQ ID NO: 1), nt 40 to 110 of Mar2 (2, SEQ ID NO:2), and Mar2 (3, SEQ ID NO:3).

The following Examples shall merely illustrate the invention. They shall not be construed, whatsoever, to limit the scope of the invention.

### EXAMPLE 1

### Colony formation

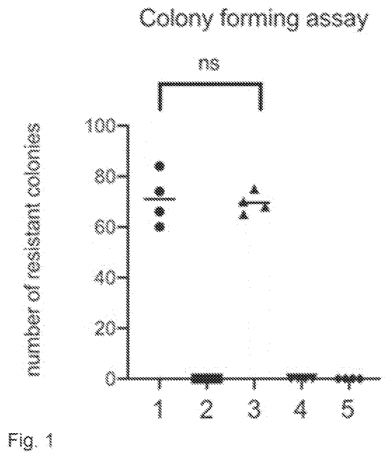

Efficiency of generating stably expressing cells was evaluated by a colony forming assay (FIG. 1). Following delivery a vector comprising a GFP reporter gene and a puromycin resistance gene into Hek293T, cells positive for GFP transgene expression were isolated via FACS sorting (FACS Aria II) and plated into a 6 cm cell culture dish. They were then cultured for 3 weeks in presence of 1 μg/ml Puromycin. After 3 weeks the cells were fixed with PFA, stained with Crystal Violet and counted. The number of colonies is considered as the efficiency of vector establishment. Vector number 3 containing the S/MAR sequence 2 (Mar2, SEQ ISD NO:3) 5′ of the GFP gene generated a number of resistant colonies comparable to the control vector carrying B-interferon S/MAR 3′ of the GFP gene, whereas vectors 2, 4 and 5 did not yield in any resistant cell.

### EXAMPLE 2

### Expression Levels

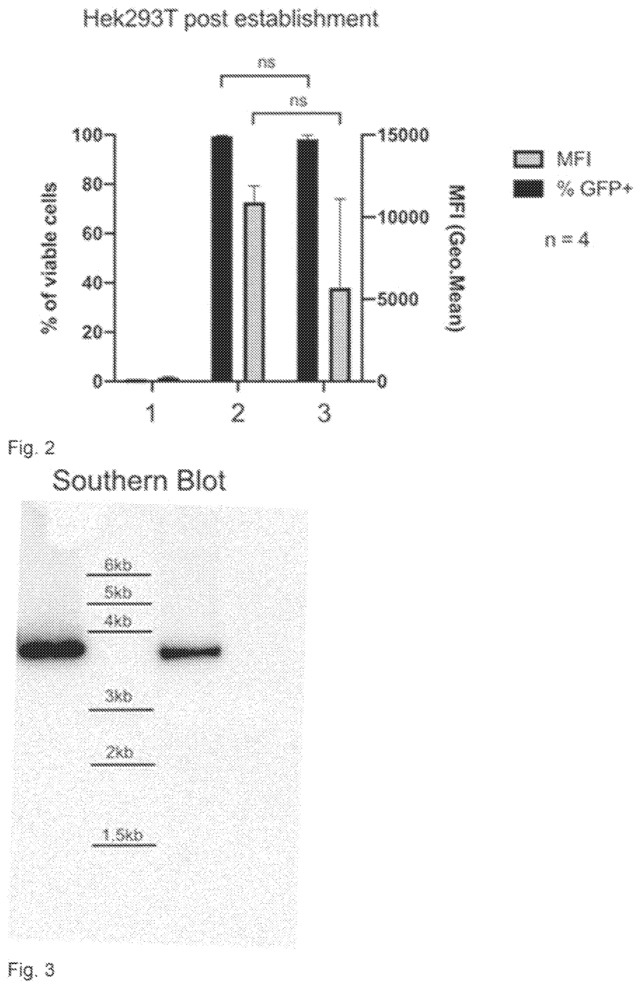

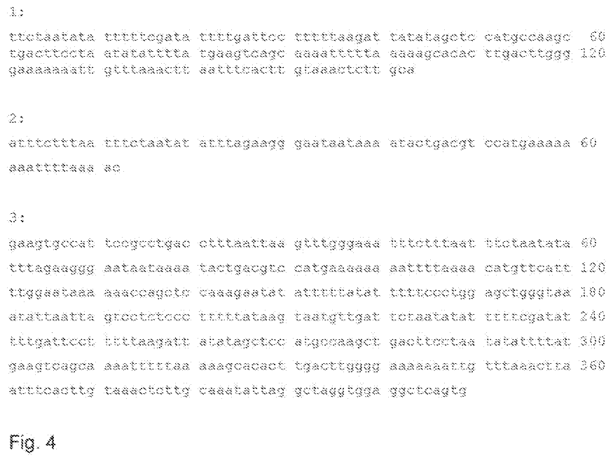

Transgene cells of Example 1 were analyzed by FACS for the relative number of cells expressing GFP (GFP+) and for the medium fluorescent intensity (MFI) of the established populations) FIG. 2). Both the control vector carrying β-interferon S/MAR 3′ of the GFP gene (2) and the vector containing the S/MAR sequence 2 5′ of the GFP gene generated cell lines in which the majority (>95%) of the cell expressed the transgene with a comparable intensity.

### EXAMPLE 3

### Episomal Maintenance

Total DNA was extracted from from Hek293T cells carrying the vector containing the S/MAR sequence 2 5′ of the GFP gene 35 days post DNA delivery, and subjected to digestion with the restriction enzyme BamHI for 12 h at 37°. The DNA fragments were resolved on a 0.8% agarose gel and transferred onto a nylon membrane. Simultaneously, plasmid DNA from the maxi preparation used to transfect the cells before establishment was treated with the same approach. The reporter gene GFP was used to generate the radioactive probe for testing the control (a), and the vector in the cell population (b) The plasmid has the same size when compared to the correspondent reference vector which demonstrate its episomal maintenance.

### LITERATURE

Geest et al. (2004), Plant Biotech J 2:13
Haase et al. (2010), BMC Biotechnology 10:20
Haase et al (2013), PLOS One 8(11):879262
Liebeich et al. (2002), NAR 30(15): 3433
Rupprecht et al. (2010), Gene 466(1-2):36
U.S. Pat. No. 6,410,314 B1
WO 2019/057773 A1
WO 2019/057774 A1
WO 2019/060253 A1
Yusufzai & Felsenfeld (2004), PNAS 101(23), 8620

1. A therapeutic cell comprising an episomal polynucleotide comprising a promoter and an expressible sequence, wherein said episomal polynucleotide further comprises an S/MAR element upstream of said promoter.

2. The therapeutic cell of claim 1, wherein said expressible sequence encodes a therapeutic gene product, preferably a therapeutic polypeptide.

3. The therapeutic cell of claim 1, wherein said expressible sequence encodes a therapeutic polypeptide, and wherein said therapeutic polypeptide is an antibody, a T Cell Receptor (TCR), a Chimeric Antigen Receptor (CAR), a cytokine, and/or a polypeptide lacking in cells affected with a genetic disease.

4. The therapeutic cell of claim 1, wherein said therapeutic polypeptide is a TCR.

5. The therapeutic cell of claim 1, wherein said therapeutic polypeptide is a CAR.

6. The therapeutic cell of claim 1, wherein said therapeutic polypeptide is a polypeptide lacking in cells affected with a genetic disease.

7. The therapeutic cell of claim 1, wherein said episomal polynucleotide further comprises a selectable marker gene.

8. The therapeutic cell of claim 1, wherein said S/MAR element comprises a nucleic acid sequence being at least 70% identical to SEQ ID NO:1.

9. The therapeutic cell of claim 1, wherein said S/MAR element comprises a nucleic acid sequence being at least 70% identical to SEQ ID NO:2.

10. The therapeutic cell of claim 1, wherein said S/MAR element comprises a nucleic acid sequence being at least 70% identical to SEQ ID NO:3.

11. The therapeutic cell of claim 1, wherein said therapeutic cell or host cell is a vertebrate cell, preferably a mammalian cell, more preferably a human cell.

12. An expression construct comprising an expressible sequence and a promoter, wherein said expression construct further comprises an S/MAR element upstream of said promoter, and wherein said expression construct replicates episomally in a mammalian cell.

13. A polynucleotide comprising an S/MAR element, wherein said S/MAR element comprises a nucleic acid sequence being at least 93%, preferably at least 95%, identical to SEQ ID NO:3.

14. The polynucleotide of claim 10, further comprising an expressible sequence and a promoter, preferably a mammalian promoter, wherein said S/MAR sequence is located upstream of said promoter.

15. (canceled)

16. (canceled)

17. (canceled)

18. (canceled)

19. (canceled)

20. (canceled)

21. A method for treating a subject suffering from cancer, autoimmune disease, and/or genetic disease, comprising contacting said subject with a therapeutic cell according to claim 1 and, thereby, treating said cancer, autoimmune disease, and/or genetic disease.

22. The method of claim 21, wherein said treating is immunotherapy.

23. The method of claim 21, wherein said genetic disease is a monogenic recessive disease, preferably is phenylketonuria, alkaptonuria, Leber's Congenital Amaurosis, Choroideremia, or Stargardt disease.


SEQUENCE LISTING (7 sequences)

SEQ ID NO: 1 - DNA, 163 bp, Artificial Sequence
  nt220-382 of Mar2
ttctaatatatttttcgatattttgattcctttttaagattatatagctccatgccaagctgacttcctaatatattttatgaagtcagcaaaatttttaaaaagcacacttgacttggggaaaaaaattgtttaaacttaatttcacttgtaaactcttgca

SEQ ID NO: 2 - DNA, 72 bp, Artificial Sequence
  nt40-110 of Mar2
atttctttaatttctaatatatttagaagggaataataaaatactgacgtccatgaaaaaaaattttaaaac

SEQ ID NO: 3 - DNA, 409 bp, Artificial Sequence
  Mar2
gaagtgccattccgcctgacctttaattaagtttgggaaatttctttaatttctaatatatttagaagggaataataaaatactgacgtccatgaaaaaaaattttaaaacatgttcattttggaataaaaaaccagctccaaagaatatatttttatatttttccctggagctgggtaaatattaattagtcctctccctttttataagtaatgttgattctaatatatttttcgatattttgattcctttttaagattatatagctccatgccaagctgacttcctaatatattttatgaagtcagcaaaatttttaaaaagcacacttgacttggggaaaaaaattgtttaaacttaatttcacttgtaaactcttgcaaatattaggctaggtggaggctcagtg

SEQ ID NO: 4 - DNA, 327 bp, Artificial Sequence
  SM1
ttccagatctcgatatctttaatttctaatatatttagaaggggtttagatttatatatgacgtccatgaaaaaaaattttaaaacgataggccagctccaaagaatatatttccctggagctggtaaatattaattagtcctctcccttctaatatatttttcgatatttttgattcctttttaagattatatagctccatgccaagctgacttcctaatatattttatgaagtcagcaaaatttttaaaaagcacacttgacttggtatatatttataaatgtttaaacttaatttcacttgtaaaactcttgcaggatccgtgc

SEQ ID NO: 5 - DNA, 370 bp, Artificial Sequence
  Mar1
gaagtgccattccgcctgacctttaattaatgggggtgtaaaaggttccaaaaaaggaaccttaataataaaatacggccacatgaatttatttatgtttgggaaatttttaatatatttttttaatgagataataaggctccacttatttattcccttttggtgtgttttgggaaaaaaatccccatgttcattttaataaacaaaaaaaatttttttcgatactttttataagtaatgttgacgatacgatacgatacaagaggagaatatatttctcctcctcttggccccaatgcctttgcaggtgtccttaccatgcgcatatcatatttatgtttagaatattaggctaggtggaggctcagtg

SEQ ID NO: 6 - DNA, 400 bp, Artificial Sequence
  Mar3
gaagtgccattccgcctgacctttaattaattattatttatgacgtccatgatttatgtttaaaaaatttaggcccattttttttataagtaatgttgacgaaccatttcacttgtaaaaaataataaaatacacttattttatttgggggtgtaaaaccctataaaaagtatagattcatatgggttttttttgcaggtgtccttgctatgacgtccatgcatgttcattttaataaataaagtttgggaaattggaaaccgtttgggaaattaaaggcacctttattttattggcttttgagaacttttgggaaaaaaataggtgtgccaagattatataccaatttatttattgacgtccatgaaaaaaaaatattaggctaggtggaggctcagtg

SEQ ID NO: 7 - DNA, 370 bp, Artificial Sequence; identical to SEQ ID NO: 5
  Mar4